(19) United States
(12) Patent Application Publication
(10) Pub. No.: US 2024/0287213 A1
(43) Pub. Date: Aug. 29, 2024

(54) NOVEL N-ALDEHYDE-FUNCTIONALIZED CHITOSAN PREPARATION METHOD AND BIOMEDICAL USES THEROF

(71) Applicant: OLIGO MEDIC INC., Laval (CA)

(72) Inventors: Abdellatif CHENITE, Kirkland (CA); Elias ASSAAD, Montréal (CA); Soudeh FAKHARI TEHRANI, Montréal (CA); Sam Alexandre SELMANI, Montréal (CA)

(21) Appl. No.: 18/650,546

(22) Filed: Apr. 30, 2024

Related U.S. Application Data
(63) Continuation of application No. PCT/CA2022/051570, filed on Oct. 25, 2022.
(60) Provisional application No. 63/276,051, filed on Nov. 5, 2021.

(51) Int. Cl. C08B 37/08; A61L 26/00

(52) U.S. Cl. CPC C08B 37/003; A61L 26/0023; A61L 26/008

(57) ABSTRACT

It is provided an aldehyde-functionalized chitosan and a method of producing same comprising reacting an amine containing polymer such as chitosan and a molecule bearing carboxyl and aldehyde functions in presence of a coupling agent, wherein the aldehyde function attaching to the polymer chain through an amide bond in absence of side reaction products.

### CROSS-REFERENCE TO RELATED APPLICATION

The present application is claiming priority from U.S. Provisional Application No. 63/276,051 filed Nov. 5, 2021, the content of which is hereby incorporated by reference in its entirety.

### TECHNICAL FIELD

It is provided a method to produce aldehyde-functionalized chitosan, where the backbone of the chitosan chain undergoes neither ring opening, nor degradation, nor undesired carboxylation.

### BACKGROUND

The modification, via targeted functionalization, of polysaccharides including chitosan is of great importance for the development of innovative materials for advanced applications. For many decades, the functionalization of chitosan has been a convenient way to improve its properties with the aim of preparing new materials with desired characteristics, such as solubility and hydrophilic character, gelling properties, and affinity toward bioactive molecules, among others. Chitosan represent a versatile biopolymer, which has been strongly recommended as a suitable modifiable material due to its excellent biocompatibility, biodegradability, no-toxicity, as well as bio adhesiveness and adsorption properties.

Aldehyde functionalization of chitosan is usually carried out through oxidation with periodate, which is the most popular method used for preparing dialdehyde of polysaccharides, such as dextran, cellulose, and alginate, among others. According to this method, the dialdehyde function is created following the attack of the periodate on the vicinal diols, promoting the cleavage of the C—C bond and the ring opening of the pyranose unit. One major drawback of such method is that it is always accompanied by side reactions yielding carboxylation and degradation products.

Aldehyde-modified polysaccharides can be reacted with amine-containing polymers, such as chitosan, to form self-healing hydrogels, which can potentially be used as tissue adhesives and sealants in the medical field. They have been proposed to close wounds, to replace sutures in internal surgical operations, and to prevent fluid leakage. Due to the high-water contents and tissue-like properties, these hydrogels are of particular interest in tissue engineering and regenerative medicine, as they can be used as scaffolds for the growth of living cells and tissues. They can also be used to encapsulate and deliver growth factors and drugs.

U.S. 2005/0002893 and U.S. Pat. No. 8,715,636 describe synthesis of dextran-aldehyde, which has been carried out by mixing sodium periodate (NaIO₄) aqueous solution (w/v) with 10% dextran aqueous solution (w/v). The concentration of the periodate solution has been varied depending on the aldehyde conversion desired. It is important to emphasize that the oxidation reaction with NaIO₄ has been found difficult to control, so that in addition to the opening of the pyranose ring, it led to extensive depolymerization and generation of carboxyl groups. To overcome this drawback, it is proposed in U.S. Pat. No. 7,247,722 a more selective oxidative method, using nitroxyl as an oxidizing agent. Despite the claimed improvements, the side reaction producing carboxyl groups cannot be avoided.

The only existing method, which ovoid the use of oxidizing agent was described in U.S. Pat. No. 4,675,394 This non-oxidative route was based on the grafting of an acetal-containing molecule onto the polysaccharide chain followed by hydrolysis of the acetal under acidic pH to make aldehyde polysaccharides.

In an effort to not after the structure of the main chain of polysaccharide, U.S. Pat. No. 8,580,950 proposed the oxidation of a polysaccharide derivative. This more complicated method consisted of a first step where polysaccharide, in this case dextran, was functionalized with given molecule followed by a second step consisting in the ozonolysis of the grafted group to create dialdehyde function. Besides the uncontrolled oxidation step, the process itself remains relatively complex and difficult to carry out.

Azevedo et al. (2012, Carbohydrate Polymers, 87: 1925-1932) reported the production of aldehyde-functionalized chitosan by reacting chitosan with nitrogen oxides generated in situ from a HNO₃/H₂PO₄NaNO₂ mixture. According to this method, the pyranose ring was preserved, but the side reactions of depolymerization and carboxylation were not avoided.

Despite the numerous modification methods proposed to date for chitosan, there remains a pressing need in the art for chitosan derivatives, which provide N-aldehyde-functionalized chitosan, wherein the aldehyde grafting is accomplished without alteration of the chitosan chain backbone, or depolymerization and without any side reaction.

### SUMMARY

It is provided a method of producing an N-aldehyde polymer comprising the step of reacting an amine containing polymer and a molecule bearing carboxyl and aldehyde functions in presence of a coupling agent, wherein the aldehyde function attaching to the polymer chain through an amide bond. In absence of side reaction products.

In an embodiment, the polymer is chitosan.

It is further provided a composition comprising an aldehyde-functionalized chitosan and a carrier, wherein said composition forms a gel in the absence of an external cross-linker.

In an embodiment, the aldehyde-functionalized chitosan comprises aldehyde groups bounded to the chitosan through an amide bond.

In another embodiment, the aldehyde groups are carboxylic acid bearing an aldehyde function.

In a further embodiment, the aldehyde-functionalized chitosan has an average molecular weight of about 1,000 to about 3,000.000 Dalton.

In an embodiment, the aldehyde-functionalized chitosan has a degree of aldehyde substitution ranging from about 5% to about 90%.

In another embodiment, the composition further comprises an amine-containing polymer forming a bio adhesive hydrogel or a sealant.

In another embodiment, the coupling agent is a water-soluble carbodimide.

In an embodiment, the water-soluble carbodiimide is 1-Ethyl-3-(3-dimethylaminopropyl)carbodiimide (EDC), Dicyclohexylcarbodiimide (DCC), N,N′-Diisopropylcarbodiimide (DIC), N-tert-butyl-N′-ethylcarbodimide (BEC), N-tert-butyl-N′-methylcarbodumide (BMC), N-Cyclohexyl-N′-isopropylcarbodiimide (CIC), or Bis[[4-(2,2-dimethyl-1,3-dioxolyl)]methyl]-carbodiimide (BDDC).

In a further embodiment, the water-soluble carbodiimide is 1-Ethyl-3-(3-dimethylaminopropyl) carbodimide (EDC).

In an embodiment, the reacting is achieved at a pH range between 6.5 and 7.5.

In another embodiment, the molecule bearing carboxyl and aldehyde is glyoxylic acid or 4-carboxybenzaldehyde.

In a further embodiment, the glyoxylic acid is glyoxylic acid sodium salt (GNa).

In an embodiment, the method encompassed further comprises solubilizing the N-aldehyde polymer in an acidic aqueous medium to form a pH sensitive solution.

In a further embodiment, the pH sensitive solution undergoes self-hydrogelation when the pH is increased to around 7.

In another embodiment, the N-aldehyde polymer is solubilized with a neutralizing agent.

In a further embodiment, the neutralizing agent is an alkaline solution.

In an embodiment, the alkaline solution is NaOH, NaHCO₃, Na₂HPO₄ or an organic base.

In a supplemental embodiment, the composition described herein is produced by the method.

In a further embodiment, the composition described herein is for use as a bio-adhesive or sealant hydrogel.

### BRIEF DESCRIPTION OF THE DRAWINGS

Reference will now be made to the accompanying drawings.

FIG. 1 illustrates the reaction pathway to produce N-aldehyde-functionalized chitosan in accordance to an embodiment.

FIG. 2a illustrates ¹H-NMR spectrum of chitosan-N-grafted glyoxylate.

FIG. 2b illustrates ¹H-NMR spectrum of chitosan-N-grafted carboxybenzaldehyde.

FIG. 3 illustrates a picture of chitosan-N-grafted glyoxylate solution at pH≈4 turned into hydrogel upon neutralization to pH≈7.

FIG. 4 illustrates rheological data recorded of chitosan-N-grafted glyoxylate in (a) solution at pH≈4, (b) monitoring of hydrogel formation after neutralization to pH≈7, and (c) frequency sweep after reaching maximum gelation (about 1 hour).

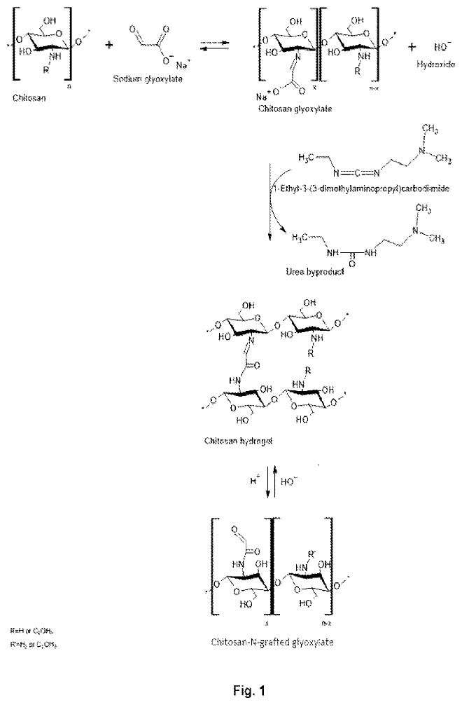

### DETAILED DESCRIPTION

It is provided a method of producing an N-aldehyde polymer comprising the step of reacting an amine containing polymer and a molecule bearing carboxyl and aldehyde functions in presence of a coupling agent, wherein the aldehyde function attaching to the polymer chain through an amide bond in absence of side reaction products.

In an embodiment, the amine-containing polymer is chitosan. Accordingly, in an embodiment, it is provided a novel chitosan derivatives bearing pendant aldehyde group. The aqueous solutions of these derivatives can undergo self-gelation under physiological pH, which make them of particular interest for the development of highly biocompatible biomedical devices for tissue engineering and regenerative medicine.

It is disclosed a novel aldehyde-functionalized chitosan (N-aldehyde chitosan), obtained by a selective reaction which causes neither opening of the pyranose ring, nor side reactions of depolymerization and carboxylation. Aqueous solutions of the resultant N-aldehyde chitosan can undergo self-hydrogelation without the need of external cross-linker or can react with amine-containing polymers to form bio adhesive hydrogels and sealants highly demanded for medical applications.

Chitosan is a linear polysaccharide obtained by alkaline deacetylation of chitin, the second most abundant natural polysaccharide after cellulose. It is composed of β-(1-4) linked 2-amino-2-deoxy-D-glucose and 2-acetamido-2-deoxy-D-glucose. Chitosan refers to chitin derivatives having a degree of deacetylation (DDA) between 50 and 100%. NMR spectroscopy is seen as the efficient method for determining the DDA, which represents the percent of acetamido transformed into amino groups. Being biocompatible, biodegradable and biologically active polysaccharide, it has been proposed for a myriad of applications in pharmaceutical and biomedical fields. Its biodegradation leads to glucosamine and N-acetylglucosamine monomers and absorbable oligosaccharides.

Accordingly, in one embodiment a composition is encompassed comprising at least one N-grafted chitosan aldehyde, having an average molecular weight of about 1,000 to about 3,000,000 Daltons and a degree of aldehyde substitution ranging from about 5% to about 90%.

The aldehyde group is bounded to the chitosan through an amide bond. Said aldehyde group is a carboxylic acid bearing an aldehyde function.

The bounding of the aldehyde group onto a chitosan chain is mediated by a coupling agent 1-Ethyl-3-(3-dimethylaminopropyl)carbodiimide (EDC), a water-soluble carbodiimide used as a carboxyl activating agent for the coupling with the primary amine of chitosan to yield amide bonds. The said coupling reaction is achieved at a pH range between 6.5 and 7.5.

As encompassed herein, the coupling agent can be a water-soluble carbodiimide such as 1-Ethyl-3-(3-dimethylaminopropyl)carbodiimide (EDC), Dicyclohexylcarbodiimide (DCC), N,N′-Diisopropylcarbodiimide (DIC), N-tert-butyl-N′-ethylcarbodiimide (BEC), N-tert-butyl-N′-methylcarbodimide (BMC), N-Cyclohexyl-N′-isopropylcarbodiimide (CIC), or Bis[[4-(2,2-dimethyl-1,3-dioxolyl)]methyl]-carbodiimide (BDDC).

The novel N-aldehyde chitosans disclosed herein are chitosans that have been chemically modified by introducing aldehyde functions onto the polymer chains. The coupling reaction used does not induce the opening of the pyranose ring and does not yield any side reaction products such as degradation and carboxylation products. As the aldehyde function is attached to the chitosan chain through an amide bond, without altering the structure of the backbone, it is expected that the aldehyde N-grafted chitosans will be more stable.

In one embodiment, the N-aldehyde chitosan is prepared by a coupling reaction between an amine-containing polymer, preferentially chitosan, and a molecule bearing both carboxyl and aldehyde functions. The coupling reaction involves the formation of amide bond between the amine and the carboxyl activated by EDC at pH comprised between 6.5 and 7.5. This leads to an aldehyde N-grafted onto the polymer chain. It is worthy to note that the optimized pH conditions established herein are essential for the efficacy of the reaction and for obtaining the right product. These pH conditions differ significantly from those recommended by the literature, generally comprised between 4 and 6.

In another embodiment, the N-aldehyde chitosan is prepared by a coupling reaction between the amine of chitosan and glyoxylic acid activated by EDC at pH comprised between 6.5 and 7.5. Moreover, what is described herein cannot be predicted by all previous works investigating chitosan-glyoxylate system. As glyoxylate forms spontaneously imine bond with chitosan, all previous works were systematically focused on reductive amination with NaBH₄, to permanently graft carboxymethyl function onto chitosan, and thus ignoring the amide formation.

In another embodiment, the aldehyde N-grafted chitosan is prepared by a coupling reaction between an amine containing polymer, preferentially chitosan, and 4-carboxybenzaldehyde activated by EDC at pH comprised between 6.5 and 7.5. The N-grafting of 4-carboxybenzaldehyde onto chitosan allow obtaining a chitosan derivative soluble in acidic aqueous media (pH-4), which turns into hydrogel upon neutralization to a pH around 7. The resulting hydrogel is expected to have tissue adhesive and sealant properties.

The N-aldehyde chitosan disclosed herein can be solubilized in acidic aqueous medium to form a pH sensitive solution (pH around 4), which undergoes self-hydrogelation when the pH is increased to neutral, around 7. Neutralizing agent is an alkaline solution prepared from NaOH, NaHCO₃. Na₂HPO₄ or an organic base, without limitation. These hydrogels may have desirable bio-adhesive and elastic properties, which make them of particular importance in biomedical and tissue engineering applications, including, but not limited to, wound closure and replacement of sutures or staples in internal surgical operations. They can also be used as sealants to prevent leakage of biological fluids such as blood, bile, gastrointestinal fluid, and ophthalmic fluids. These biocompatible hydrogels may also be suitable to provide scaffolding matrices for living cells and tissues to address some unmet needs in regenerative medicine.

The N-aldehyde chitosan disclosed herein may be also used. In place of oxidized polysaccharide to react with unmodified chitosan or with a multi-arm polyether amine or polyvinyl alcohol having amino groups, to produce bio-adhesive and sealant hydrogels.

### Example I

### Synthesis of N-glyoxylate chitosan

In a typical experiment, 10.00 g of chitosan, having DDA of 90% and high molecular weight, was dispersed in 1.0 L of deionized water. While stirring, add 54.4 mL of HCl solution (1M). When the chitosan is completely dissolved at room temperature, add 15.6 g of glyoxylic acid sodium salt (GNa). Then, 54.4 mL of NaHCO₃ solution (0.5M) was added and followed by addition of 54.4 mL of NaOH solution (0.5M) under vigorous stirring. Once the mixture was clear and the pH value is around 7.2, a 13.03 g of EDC solubilized in 100 mL of deionized water was added to the mixture under vigorous stirring. The agitation was continued for at least 20 minutes to ensure a good homogenization of the final mixture, which turned into clear hydrogel, a sign that N-grafting of glyoxylate had taken place. Then, the resultant hydrogel was incubated at 50° C. for 20 hours to allow further N-grafting of glyoxylate.

The resultant hydrogel was redissolved in acid and the modified polymer free of unreacted glyoxylic acid was reprecipitated by adding isopropanol. A cycle of dissolution in water followed by reprecipitation. In isopropanol was operated twice to ensure complete cleaning of the modified polymer.

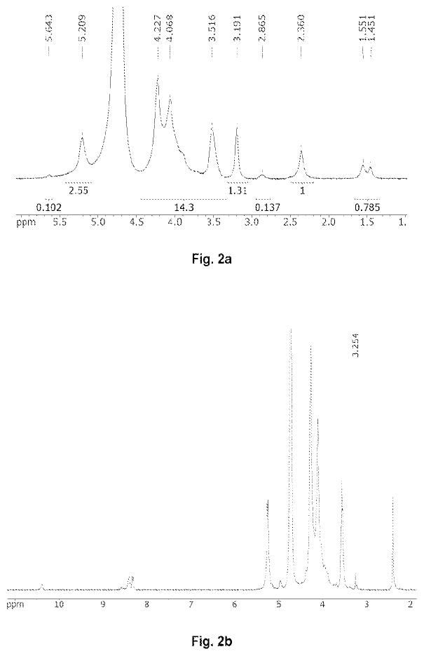

Proton-NMR performed on the obtained product is shown on FIG. 2a. As the new peak at 3.19 ppm has been assigned to the hydrogen of aldehyde function (CHO), the degree of substitution has been evaluated to about 39.7%.

### Example II

### Synthesis of N-carboxybenzaldehyde chitosan

In a typical experiment, 5.00 g of chitosan, having DDA of 90% and high molecular weight, was dispersed in 500 mL of deionized water. While stirring, add 27.2 mL of HCl solution (1M). When the chitosan is completely dissolved at room temperature, 27.2 mL of NaHCO₃ solution (0.5M) is added, necessary amount to increase the pH in the vicinity of 6.5. Then, add 1.02 g of 4-carboxybenzaldehyde under vigorous stirring. Then, while maintaining stirring, add 1.54 g of EDC solubilized in 15 mL of deionized water to the mixture. The agitation was continued for at least 20 minutes to ensure a good homogenization of the final mixture. Then, the mixture was incubated at 50° C. for about 20 hours to allow its gelation.

The resultant hydrogel was redissolved in acid and the modified polymer free of unreacted carboxybenzaldehyde was reprecipitated in isopropanol. Additional 2 operations of dissolution in water followed by reprecipitation in isopropanol were necessary to dean the N-aldehyde-modified polymer.

The pure product Chitosan-N-grafted carboxybenzaldehyde in powder form may be solubilized in aqueous media at pH≈4, and when neutralized to pH≈7, it turns into clear hydrogel.

The degree of substitution has found around 10% according to ¹H-NMR spectrum shown in FIG. 2b.

### Example III

### PH-Sensitive Gelation of N-Aldehyde Chitosan

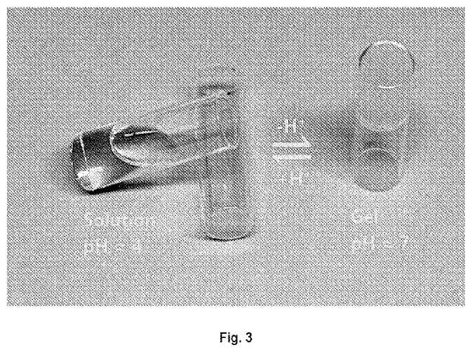

Aldehyde-bearing chitosan, chitosan-N-grafted glyoxylate was solubilized in water at 100% protonation (pH≈4). The solution was neutralized by mixing NaHCO₃ 0.82M in an amount to match stoichiometrically the amines of the aldehyde-bearing chitosan. This results in nearly neutral mixture (pH≈7), which turned rapidly in clear and elastic hydrogel. The transition from solution to hydrogel can be visually observed as shown by the image of FIG. 3.

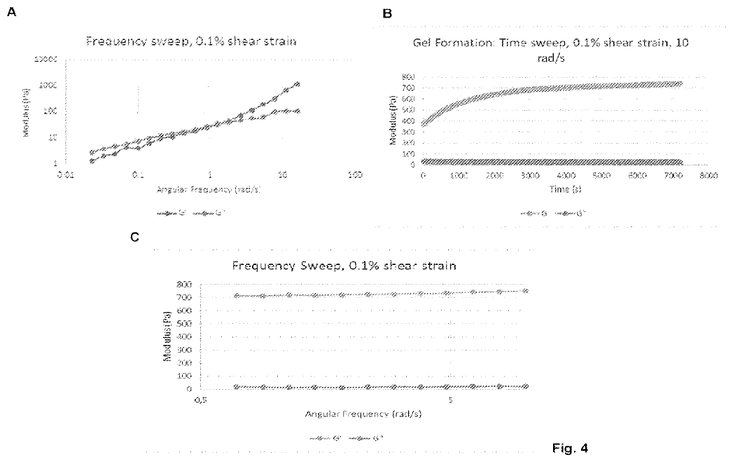

The gelation has been also monitored by rheometer. The graphics of FIG. 4, represent the rheological data recorded for chitosan-N-grafted glyoxylate solution (pH≈4) (FIG. 4a), and chitosan-N-grafted glyoxylate hydrogel (pH≈7) (FIGS. 4b, 4c and 4d).

While the present disclosure has been described in connection with specific embodiments thereof, it will be understood that it is capable of further modifications and this application is intended to cover any variations, uses, or adaptations and including such departures from the present disclosure as come within known or customary practice within the art and as may be applied to the essential features hereinbefore set forth, and as follows in the scope of the appended claims.

1: A composition comprising an aldehyde-functionalized chitosan and a carrier, wherein said composition forms a gel in the absence of an external cross-linker.

2: The composition of claim 1, wherein the aldehyde-functionalized chitosan comprises aldehyde groups bounded to the chitosan through an amide bond.

3: The composition of claim 2, wherein said aldehyde groups are carboxylic acid bearing an aldehyde function.

4: The composition of claim 1, wherein the aldehyde-functionalized chitosan has an average molecular weight of about 1,000 to about 3,000,000 Dalton.

5: The composition of claim 1, wherein the aldehyde-functionalized chitosan has a degree of aldehyde substitution ranging from about 5% to about 90%.

6: The composition of claim 1, further comprising an amine-containing polymer forming a bio adhesive hydrogel or a sealant.

7: A method of producing an N-aldehyde polymer comprising the step of reacting an amine containing polymer and a molecule bearing carboxyl and aldehyde functions in presence of a coupling agent, wherein the aldehyde function attaching to the polymer chain through an amide bond in absence of side reaction products.

8: The method of claim 7, wherein the polymer is chitosan.

9: The method of 7, wherein the coupling agent is a water-soluble carbodiimide.

10: The method of claim 9, wherein the water-soluble carbodiimide is 1-Ethyl-3-(3-dimethylaminopropyl)carbodiimide (EDC), Dicyclohexylcarbodiimide (DCC), N,N′-Diisopropylcarbodiimide (DIC), N-tert-butyl-N′-ethylcarbodiimide (BEC), N-tert-butyl-N′-ethylcarbodiimide (BMC), N-Cyclohexyl-N′-isopropylcarbodiimide (CIC), or Bis[[4-(2,2-dimethyl-1,3-dioxolyl)]methyl]-carbodiimide (BDDC).

11: The method of claim 9, wherein the coupling agent is 1-Ethyl-3-(3-dimethylaminopropyl)carbodiimide (EDC).

12: The method of claim 7, wherein the reacting is achieved at a pH range between 6.5 and 7.5.

13: The method of claim 7, wherein the molecule bearing carboxyl and aldehyde is glyoxylic acid or 4-carboxybenzaldehyde.

14: The method of claim 13, wherein the glyoxylic acid is glyoxylic acid sodium salt (GNa).

15: The method of claim 7, further comprising solubilizing the N-aldehyde polymer in an acidic aqueous medium to form a pH sensitive solution.

16: The method of claim 15, wherein the pH sensitive solution undergoes self-hydrogelation when the pH is increased to around 7.

17: The method of claim 15, wherein the N-aldehyde polymer is solubilized with a neutralizing agent.

18: The method of claim 17, wherein the neutralizing agent is an alkaline solution.

19: The method of claim 18, wherein the alkaline solution is NaOH, NaHCO₃, Na₂HPO₄ or an organic base.

20: The composition of claim 1, produced by the method of claim 7.

21. (canceled)
